(19) United States
(12) Patent Application Publication
(10) Pub. No.: US 2010/0317727 A1
(43) Pub. Date: Dec. 16, 2010

(54) ROSEMARY EXTRACTS, DIETARY AND PHARMACEUTICAL COMPOSTIONS CONTAINING THEM AND THEIR USES

(76) Inventors: Antoine De Saizieu, Brunstatt (FR); Ann Fowler, Rheinfelden (CH); Regina Goralczyk, Grenzach-Wyhlen (DE); Claus Kilpert, Mannheim (DE); Goede Schueler, Eimeldingen (DE)

Correspondence Address: NIXON & VANDERHYE, PC, 901 NORTH GLEBE ROAD, 11TH FLOOR, ARLINGTON, VA, 22203, US

(21) Appl. No.: 12/515,320

(22) PCT Filed: Nov. 22, 2007
(86) PCT No.: PCT/EP07/10135

§ 371 Date: May 18, 2009

(51) Int. Cl. A61K 31/352; A61P 25/00; A61P 25/24; A61P 25/18; A61P 25/20

(52) U.S. Cl. 514/453

(57) ABSTRACT

The present invention refers to rosemary extracts for use as medicament for the treatment of a disorder connected to reduced neurotransmission, as well as to dietary and pharmaceutical compositions containing such rosemary extracts and their uses.

### BRIEF DESCRIPTION OF THE INVENTION

The present invention refers to rosemary extracts for use as medicaments, especially for the treatment of disorders connected to impaired, i.e. reduced, neurotransmission, as well as to dietary and pharmaceutical compositions containing such rosemary extracts and their uses.

### BACKGROUND OF THE INVENTION

It is well known that impaired neurotransmission, e.g. low neurotransmitter levels, is connected to mental diseases, such as depression and generalised anxiety disorder (GAD), and increased susceptibility to stress.

Compounds that increase neurotransmitter levels in the brain and thus enhance their transmission, can exhibit antidepressant properties as well as beneficial effects on a variety of other mental disorders (Neurotransmitters, drugs and brain function, R. A. Webster (ed), John Wiley & Sons, New York, 2001, p. 187-211, 289-452, 477-498). The main neurotransmitters are serotonin, dopamine, noradrenaline (=norepinephrine), acetylcholine, glutamate, gamma-amino-butyric acid, as well as neuropeptides. Those neurotransmitters of particular relevance to mood-related disorders include serotonin, noradrenaline and dopamine. Enhanced or prolonged neurotransmission is achieved by increasing the concentration of the neurotransmitter in the synaptic cleft, through inhibition of re-uptake into the pre-synaptic nerve ending, or by preventing neurotransmitter catabolism by inhibition of degrading enzymes such as monoamine oxidase (MAOs)-A and -B.

### Antidepressants and Mood-Related Disorders

Tricyclic antidepressant compounds (TCAs), such as imipramine, amitriptyline, and clomipramine, inhibit the re-uptake of serotonin and noradrenaline. They are widely regarded as among the most effective antidepressants available, but they have a number of disadvantages because they additionally interact with muscarinic acetylcholine-, histamine- and serotonin-receptors. Side effects resulting from such activities include dry mouth, blurred vision, constipation and urinary retention, in addition to postural hypotension. Most importantly, TCAs are not safe when taken in overdose, frequently showing acute cardiotoxicity.

Another class of antidepressant drugs are the so-called SSRIs (selective serotonin re-uptake inhibitors), including fluoxetine, paroxetine, sertraline, citalopram and fluvoxamine, that block the serotonin transporter (SERT), a high affinity sodium chloride-dependent neurotransmitter transporter that terminates serotonergic neurotransmission by re-uptake of serotonin. They have been proven as effective in the treatment of depression and anxiety as TCAs, but are usually better tolerated. These medications are typically started at low dosages and are increased until they reach a therapeutic level. A common side effect is nausea. Other possible side effects include decreased appetite, dry mouth, sweating, infection, constipation, tremor, yawning, sleepiness and sexual dysfunction.

In addition, compounds that prevent the catabolism of neurotransmitters more broadly by inhibiting MAOs-A and -B exhibit antidepressant effects. MAOs catalyse the oxidation of amine group-containing neurotransmitters, such as serotonin, noradrenaline and dopamine.

Furthermore, modulators of neurotransmission exert pleiotropic effects on mental and cognitive functions.

There is a need for compounds for the treatment or prevention of mental diseases and/or disorders which do not show the negative side effects of known antidepressants. Many patients are interested in alternative therapies which could minimise the side-effects associated with high doses of drugs and yield additive clinical benefits. Severe depression is a long-lasting and recurring disease, which is usually poorly diagnosed. Furthermore many patients suffer from mild or moderately severe depression. Thus, there is an increasing interest in the development of compounds, as well as pharmaceutical and/or dietary compositions, that may be used to treat mental diseases/disorders or to prevent the development of mental diseases/disorders, such as depression, in people at risk, and to stabilise mood and achieve emotional balance.

Patients often suffer either as comorbidity to depression, or alone, from GAD, which is a highly prevalent anxiety condition and chronic illness in primary care (˜10% of patients) (Wittchen et al 2005. Eur. Neuropsycho. 15:357-376). Patients present themselves to their primary care physician with multiple physical symptoms. GAD is characterised by chronic tension, and anxious worrying and tension (>6 months), which are disabling and uncontrollable, and accompanied by a characteristic hypervigilance syndrome (including restlessness, muscle tension and sleep problems). If untreated, GAD runs a chronic, fluctuating course and tends to become increasingly severe with age. GAD patients suffer from subsyndromal depression and contribute to the highest overall direct and indirect health economic burden of all anxiety and depressive disorders. Despite high GAD incidence, few sufferers are diagnosed, prescribed medication, or receive psychiatric referral; simple diagnostic tools to aid patient recognition and monitoring are needed. Regardless of specific diagnosis, physicians require effective GAD-symptom treatments. SSRIs, such as paroxetine, are effective for GAD treatment (Stocchi et al., 2003 J. Clin. Psych., 63(3): 250-258). Also, systematic reviews and placebo-controlled RCTs (Randomised Clinical Trials) indicate that some SSRIs (escitalopram, paroxetine and sertraline), the SNRI (Selective Noradrenaline Reuptake Inhibitor) venlafaxine, some benzodiazepines (alprazolam and diazepam), the TCA imipramine, and the 5-HT_{1A} partial agonist, buspirone, are all efficacious in acute treatment. In general, the effect of treatment is often moderate and symptoms reappear when the treatment period is discontinued. Therefore, a continuous long-term treatment or prevention with compounds which have fewer side effects than SSRIs and can be taken over long time periods might be favourable over drug treatment.

Mood disorders and occupational stress can lead to sleep disorders, insomnia, low sleep quality and general disturbances in circadian rhythms (so-called biorhythms); such conditions are often chronic and persistent in nature. Also, dysregulation of circadian rhythms induced by long-haul flights (jet-lag) and shift-work can cause similar symptoms and distress. Therefore, treatment with dietary supplementation to maintain a normal circadian rhythm (that a human or animal is used to) and/or to alleviate and prevent symptoms associated with a disturbed circadian rhythm, such as impairment of cognitive function and memory and mental and physical fatigue, thus improving the overall quality of life and benefiting the vital energy of a person in need thereof, would be most desirable.

### DETAILED DESCRIPTION OF THE INVENTION

### Rosemary Extracts and their Preferred Components

It has been found, in accordance with this invention, that rosemary extracts can be used in medicaments for the treatment of a disorder connected to reduced neurotransmission. In particular, this demand is met by those rosemary extracts that are manufactured by a process comprising the following steps:
grinding dried leaves of Rosmarinus officinalis,
extracting the ground rosemary leaves with acetone to obtain an acetone extract,
optionally (preferably) filtrating the acetone extract,
concentrating the acetone extract by (partly, preferably nearly completely) removing the acetone,
optionally (preferably further) spray-drying the thus concentrated acetone extract.

The thus manufactured rosemary extract contains >35 weight-% of carnosic acid, preferably >40 weight-% of carnosic acid, preferably >50 weight-% of carnosic acid, based on the total weight of the rosemary extract. Thus manufactured rosemary extracts are especially suitable for the purposes of the present invention.

Rosemary extracts especially suitable for the purposes of the present invention contain
rosmanol, preferably in the range of from 0.05 to 3 weight-%, more preferably in the range of from 0.1 to 1.0 weight-%, most preferably in the range of from 0.2 to 0.7 weight-%,
carnosol, preferably in the range of from 0.5 to 7.0 weight-%, more preferably in the range of from 1 to 5.0 weight-%, most preferably in the range of from 2.5 to 4.0 weight-%,
carnosic acid, preferably in the range of from 20 to 65 weight-%, more preferably in the range of from 25 to 60 weight-%, most preferably in the range of from 30 to 55 weight-%; and
carnosic acid 12-methyl ether, preferably in the range of from 1 to 20 weight-%, more preferably in the range of from 5 to 15 weight-%, most preferably in the range of from 7 to 12 weight-%,
   based on the total weight of the rosemary extract and measured by HPLC-UV (High Performance Liquid Chromatography-Ultraviolet) at 210 nm with the pure substances as reference.

“Rosmanol” means the racemic mixture as well as pure (4aR,9S,10aS)-rosmanol or pure (4aS,9R,10aR)-rosmanol or any mixture or diastereoisomer of them. (4aR,9S,10aS)-rosmanol is preferred.

“Carnosol” means the racemic mixture as well as pure (4aR,9S,10aS)-carnosol or pure (4aS,9R,10aR)-carnosol or any mixture or diastereoisomer of them. (4aR,9S,10aS)-carnosol is preferred.

“Carnosic acid” means the racemic mixture as well as pure (4aR,10aS)-carnosic acid or pure (4aS,10aR)-carnosic acid or any mixture or diastereoisomer of them. Preferred is (4aR,10aS)-carnosic acid.

“Carnosic acid 12-methyl ether” means the racemic mixture as well as pure (4aR,10aS)-carnosic acid 12-methyl ether or pure (4aS,10aR)-carnosic acid 12-methyl ether or any mixture or diastereoisomer of them. Preferred is (4aR,10aS)-carnosic acid 12-methyl ether.

Uses of Rosemary Extracts and their Components

Thus, in one aspect the invention relates to rosemary extracts, for use as medicaments for the treatment of a disorder connected to reduced neurotransmission.

In another aspect, the invention relates to the use of rosemary extracts for the manufacture of a composition for the treatment of a disorder connected to reduced neurotransmission, particularly for the manufacture of an antidepressant, a mood/vitality improver, a stress reliever, a condition improver, a reducer of anxiety, a reducer of obsessive-compulsive behaviour, a relaxant, a sleep improver and/or an insomnia alleviator.

In still another aspect, the invention relates to a dietary composition comprising a rosemary extract, as defined above, as well as to a pharmaceutical composition comprising a rosemary extract, as defined above, and a conventional pharmaceutical carrier.

Furthermore, the invention relates to a method for the treatment of a disorder connected to reduced neurotransmission in animals including humans, said method comprising administering an effective dose of a rosemary extract as defined above to animals including humans which are in need thereof.

Animals in the context of the present invention include humans and encompass mammals, fish and birds. Preferred “animals” are humans, pet and companion animals and farm animals. Examples of pet and companion animals are dogs, cats, birds, aquarium fish, guinea pigs, (jack) rabbits, hares and ferrets. Examples of farm animals are fish, pigs, horses, ruminants (cattle, sheep and goats) and poultry.

In the context of this invention “treatment” also encompasses co-treatment as well as prevention. “Prevention” can refer to either the first occurrence (primary prevention) or to a recurrence (secondary prevention).

The term “reduced neurotransmission” is used in the present application in accordance with its meaning well-known to the person skilled in the art and relates to a dysregulation of neurotransmission and which may occur at the level of neurotransmitter biosynthesis, processing, storage, release, re-uptake and receptor binding. Reduced neurotransmission may manifest itself in animals including humans as a disturbance of behaviour, emotions, mood and thinking processes, for example, in one of various types of depression.

Thus, the present invention is also directed to a method for the prevention of a disorder connected to reduced neurotransmission in animals including humans, said method comprising administering an effective amount of a rosemary extract, with the preferences as described above, to animals including humans which are in need thereof. In this regard an effective amount of a rosemary extract may especially be used for maintaining mental well-being, for maintaining a balanced cognitive function, for helping to reduce the risk of mood swings, for helping to retain a positive mood and for supporting cognitive wellness, and for helping to maintain a good sleep quality.

In the context of this invention the term “disorder” also encompasses diseases.

Medicaments for the treatment of disorders connected to reduced neurotransmission encompass antidepressants, mood/vitality improvers, stress relievers, condition improvers, anxiety reducers and obsessive-compulsive behaviour reducers, relaxants, sleep improvers and/or insomnia alleviators. They all improve, enhance and support neurotransmission, especially in the central nervous system, and therefore alleviate mental dysfunction.

Antidepressants are medicaments/compositions for treating mental-, behavioural- and emotional/affective-, neurotic-, neurodegenerative-, eating- and stress-related-disorders, such as unipolar depression, bipolar depression, acute depression, chronic depression, subchronic depression, dysthymia, postpartum depression, premenstrual dysphoria/syndrome (PMS), climacteric depressive symptoms, aggression, attention deficit disorders, social anxiety disorders, seasonal affective disorders and anxiety (disorders), such as GAD, fibromyalgia syndrome, post-traumatic stress disorders, panic disorders and obsessive compulsive disorders, restless leg syndrome, nervousness, migraine/primary headaches and pain in general, emesis, bulimia, anorexia nervosa, binge eating disorder, gastrointestinal disorders, burn-out syndrome and irritability.

Antidepressants can also be used for (the manufacture of compositions for) primary and secondary prevention and/or the treatment of neurocognitive impairment. Furthermore they are also effective in the treatment of depressive symptoms or other symptoms related to disturbed neurotransmission occurring as comorbidity in chronic diseases such as cardiovascular diseases, strokes, cancer, Alzheimer's disease, Parkinson's disease, and others.

The rosemary extracts as defined above, as well as dietary/pharmaceutical compositions containing them, are thus suitable for the treatment of animals including humans.

In a further embodiment of the present invention, rosemary extracts as defined above find use as mood improvers in general as well as for the manufacture of compositions for such use (dietary/pharmaceutical compositions). “Mood improver”, “emotional wellness booster” or “vitality improver” means that the mood of a person treated with it is enhanced, that self-esteem is increased and/or that negative thoughts are reduced. It also means that the emotions are balanced and/or that general, especially mental, well-being and vitality is improved or maintained, as well as that the risk of mood swings is (helped to be) reduced and that a positive mood is (helped to be) retained.

Rosemary extracts as defined above can also be used in general as anxiety reducers and/or obsessive-compulsive behaviour reducers for animals including humans; preferably for humans, pet animals and farm animals.

“Anxiety reducer” means that chronic tension and anxious worrying and tension are lessened or alleviated. Hypervigilance syndrome, including restlessness, muscle tension and sleep problems, are reduced or relieved. Social- and other phobias are resolved. In general, the social environment is experienced as less threatening. The person is emotionally relaxed, experiences comfort and enjoys company and contact with other people.

“Relaxant”, “sleep improver” or “insomnia alleviator” means improving sleep onset and helping a person to easily enter sleep, to maintain undisrupted sleep throughout the night. It also means that circadian rhythm-associated sleep disturbances, due to jet-lag or shift work, are corrected and symptoms associated with sleeplessness, i.e. impairment of cognitive function and memory, mental and physical fatigue, dreaminess, are abolished or relieved and the overall quality of life and vital energy are improved.

Moreover, rosemary extracts as defined above, as well as compositions comprising an effective dose of them, are useful for the treatment, prevention and alleviation of stress-related symptoms, for the treatment, prevention and alleviation of symptoms related to work overload, exhaustion and/or burn-out, for the increase of resistance or tolerance to stress and/or to favour and facilitate relaxation in normal healthy individuals i.e. such compositions have an effect as “stress relievers”.

A further embodiment of the present invention relates to the use of rosemary extracts as defined above, and to the use of compositions containing them (dietary/pharmaceutical compositions), as “condition improvers”, i.e. as means to reduce irritability and tiredness, to reduce, prevent or alleviate physical and mental fatigue and to increase energy in more general terms, especially to increase brain energy production, in diseased or normal healthy individuals.

They are also useful for cognition improvement in general, for the regulation of hunger and satiety as well as for the regulation of motor activity.

The present invention not only refers to rosemary extracts as defined above and their compositions (dietary/pharmaceutical compositions containing them) for use as medicaments, especially for the treatment of disorders connected to reduced neurotransmission, but also for the methods for the treatment of such disorders themselves, as already mentioned above.

### Veterinary Uses

Pets and farm animals can be in conditions in need of enhanced or improved neurotransmission, which can be provided by the present invention. Animals may exhibit adverse behavioural and/or physiological reactions to stressful situations; animals raised in mass production environments, or being transported under unfavourable conditions, can display a decline in meat or milk quantity or quality; stressed poultry can resort to feather-picking, reduced egg laying and cannibalism. Many animals can become aggressive or display stereotypic-, anxiety- and obsessive-compulsive-behaviours under adverse housing or transport conditions.

Thus, another aspect of this invention is veterinary uses of rosemary extracts as defined above, as dietary/pharmaceutical compositions.

In a preferred embodiment of the present invention, rosemary extracts as defined above are administered for preventing stress in farm animals and mass production livestock husbandry, during transport to slaughter and/or for preventing loss of quality of meat of said farm animals under such circumstances. The farm animals are preferably poultry, cattle, sheep, goats and swine.

In another preferred embodiment of the present invention, rosemary extracts, as defined above, are administered to poultry for preventing feather-picking and cannibalism resulting in, for example, loss of meat quality and egg production.

Another aspect of this invention is a method for preventing and/or alleviating stress in aquaculture, comprising administering rosemary extracts as defined above to animals which are in need thereof, wherein the animals are fish or shrimps.

In another preferred embodiment of the present invention, rosemary extracts, as defined above, are administered to pets or companion animals for reduction of stress, tension and aggressiveness and compulsive behaviour exhibited under stressful conditions, such as separation, change or loss of owner, during holiday separation and husbandry in so-called “animal hotels” and husbandry in animal shelters or refuges.

Still another aspect of this invention is a method for preventing/reducing symptoms associated with stressful conditions in animals used in the fur industry, preferably minks, foxes and hares.

### Preferred Doses and Compositions of the Present Invention

For humans a suitable daily dosage of rosemary extract for the purposes of the present invention may be within the range of from 0.001 mg per kg body weight to 100 mg per kg body weight per day. More preferred is a daily dosage in the range of from 0.01 to 10 mg per kg body weight, and especially preferred is a daily dosage in the range of from 0.05 to 5.0 mg per kg body weight.

The term “dietary compositions” comprises any type of (fortified) food/feed and beverages, also including clinical nutrition and dietary supplements. The dietary compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Beside a pharmaceutically acceptable carrier and a rosemary extract as defined above, the pharmaceutical compositions according to the present invention may further contain conventional pharmaceutical additives and adjuvants, excipients or diluents, including, but not limited to, water, gelatine of any origin, vegetable gums, ligninsulfonate, talc, sugars, starch, gum arabic, vegetable oils, polyalkylene glycols, flavouring agents, preservatives, stabilisers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administering to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid forms such as solutions, emulsions or suspensions as e.g. beverages, pastes and oily suspensions. The pastes may be filled into hard or soft shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Examples for fortified food are cereal bars and bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, teas and milk-based drinks. Liquid foods are e.g. soups and dairy products (e.g. muesli drinks).

In solid dosage unit preparations for humans, the rosemary extracts are suitably present in an amount in the range of from 0.1 mg to 1000 mg, preferably in the range of from 1 mg to 500 mg per dosage unit.

In dietary compositions, especially in food and beverages for humans, the rosemary extracts are suitably present in an amount in the range of from 0.0001 (1 mg/kg) to 5 weight-% (50 g/kg), preferably in the range of from 0.001 (10 mg/kg) to 1 weight-% (10 g/kg), more preferably in the range of from 0.01 (100 mg/kg) to 0.5 weight-% (5 g/kg), based upon the total weight of the food or beverage.

In food and drinks in a preferred embodiment of the invention the amount of rosemary extracts are in the range of from 10 to 30 mg per serving, i.e. ca. 120 mg per kg food or drink.

For animals excluding humans a suitable daily dosage of rosemary extracts, for the purposes of the present invention may be within the range of from 0.001 mg per kg body weight to 1000 mg per kg body weight per day. More preferred is a daily dosage in the range of from 0.1 mg to 500 mg per kg body weight, and especially preferred is a daily dosage in the range of from 1 mg to 100 mg per kg body weight.

The invention is illustrated further by the following examples.

### EXAMPLES

### Example 1

### Preparation of the Rosemary Extract

Dried leaves of Rosmarinus officinalis were ground and then extracted with acetone. The thus obtained acetone extract was filtered and concentrated by removing the acetone. The concentrated acetone extract was then spray-dried.

The thus produced rosemary extract (“Oxy'less” (No. 993 369) from Naturex, Avignon, France) contained:

0.5 weight-% of rosmanol,
3.3 weight-% of carnosol,
36.7 weight-% of carnosic acid, and
9.0 weight-% of carnosic acid 12-methyl ether,
based on the total weight of the rosemary extract and measured by HPLC-UV at 210 nm with the pure substances as reference.

The amount of each component contained within specific doses of the rosemary extract used for in vivo efficacy studies (Examples 4 and 5) were as follows:

| | % present | | | | | |
|---|---|---|---|---|---|---|
| Compound/extract | (w/w) | Dose administered (mg/kg) | | | | |
| total extract | — | 27 | 81 | 272 | 409 | 545 |
| rosmanol | 0.5 | 0.14 | 0.4 | 1.4 | 2 | 2.7 |
| carnosol | 3.3 | 0.9 | 2.7 | 9 | 13.5 | 18 |
| carnosic acid | 36.7 | 9.9 | 29.7 | 100 | 150 | 200 |
| carnosic acid-12-methyl- | 9.0 | 2.4 | 7.3 | 24.5 | 36.8 | 49 |
| ether | | | | | | |

### Example 2

### Serotonin Uptake Inhibition by Rosemary Extract

Human embryonic kidney (HEK-293) cells stably expressing the human serotonin re-uptake transporter (hSERT) were obtained from R. Blakely, Vanderbilt University, USA. The cells were routinely grown in Dulbecco's Modified Eagle's Medium (Bioconcept) containing 10% dialysed foetal calf serum (Invitrogen), penicillin, streptomycin, L-glutamine and the antibiotic G418 and passaged by trypsinisation. On the day of assay, cells from 80% confluent flasks were harvested by gentle washing with warm phosphate-buffered saline (PBS). Cells were then washed once by centrifugation and re-suspended in Krebs-Ringer bicarbonate buffer (Sigma) supplemented with 35 μM pargyline, 2.2 mM CaCl₂, 1 mM ascorbic acid and 5 mM N-2-hydroxyethylpiperazine-N′-2-ethanesulfonic acid (HEPES buffer) at a concentration of 10,000 cells in 160 μl of buffer, and aliquoted into round-bottomed polypropylene 96-well microtitre plates (Corning) at 10,000 cells per well. Serotonin uptake into the cells was determined by addition of radio-labelled [³H]-serotonin (GE Healthcare) at a concentration of 20 nM, and incubation for 40 minutes at 37° C. with gentle shaking. At the end of this time unincorporated label was removed by filtration though Unifilter 96 GF/B plates (Perkin Elmer) using a Tomtec Mach III M cell harvester. The incorporated serotonin retained on the plates was quantified by liquid scintillation counting using Microscint-40/Topcount (Perkin Elmer).

The effect of the rosemary extract, obtained according to Example 1, on serotonin uptake was determined by its inclusion in the assay at a range of concentrations between 0.03 and 100 μg/ml for 10 minutes prior to and during the addition of [³H]-serotonin. The SSRI, fluoxetine, (0.03 nM-1 μM) was used as reference compound. Serotonin uptake via the transporter was inhibited by the rosemary extract in a concentration-dependent manner.

The calculated IC₅₀ values for inhibition of serotonin uptake by the rosemary extract and fluoxetine are shown in Table 1.

TABLE 1
Inhibition of serotonin uptake into transfected HEK-293 cells by
rosemary extract and fluoxetine. Data is shown as mean ± s.e.m.,
where multiple assays were performed.

| | IC₅₀ for Tritiated Serotonin | |
|---|---|---|
| Substance | Uptake | |
| Rosemary Extract (according to Example 1) | 6.9 ± 1.4 | μg/ml (n = 2) |
| Fluoxetine | 5 | nM |

### Example 3

### Monoamine Oxidase Inhibition by Rosemary Extract

The organic amines p-tyramine or benzylamine were used as substrates for the monoamine oxidase A (MAO-A) and B (MAO-B) enzymes respectively. The H₂O₂ produced by this reaction was quantified by reaction with vanillic acid, catalysed by horse radish peroxidase (HRP).

The reactions were carried out at 37° C. in polystyrene microtitre plates. The MAO enzymes (final concentration 2 U/ml) were mixed with either p-tyramine (Sigma, final concentration 0.5 mM) or benzylamine (Sigma, final concentration 0.5 mM) as appropriate and the chromogenic solution (containing vanillic acid (Fluka), 4-aminoantipyrine (Fluka) and horse radish peroxidase (Sigma), final concentrations 0.25 mM, 0.125 mM and 1 U/ml respectively) in 0.2 M potassium phosphate buffer, pH 7.6. After 40 min incubation, plates were analysed in a microtitre plate absorbance reader e.g. Spectramax M5 (Molecular Devices Corporation), at 495 nm.

The effect of the rosemary extract on the monoamine oxidase enzymes was determined by its inclusion in the assay at a range of concentrations between 0.03 and 100 μg/ml for 10 minutes prior to and during the incubation with substrate. To determine the effect of the extracts on the HRP-catalysed portion of the reaction, the MAO enzyme was replaced by H₂O₂ (Molecular Probes, final concentration 50 μM). The reactions containing MAO-A and MAO-B were both inhibited by the rosemary extract in a concentration-dependent manner, whilst the control reaction, was unaffected.

The measured IC₅₀ values for inhibition of monoamine oxidase activity by the rosemary extract were 0.93±0.13 μg/ml and 3.0±0.4 μg/ml, for MAO-A and MAO-B, respectively (n=2, in each case).

### Example 4

### Effects of Rosemary Extract on Behaviour in Porsolt's Forced Swim Test

The Forced Swim Test (FST) was first reported in 1977 for screening of antidepressant-like compounds in rats (Porsolt et al 1977 Nature, 266:730-732) and, later, modified for testing mice (Porsolt et al 1977 Arch. Int. Pharmacodynamie, 229:327-336). “Behavioural despair” was demonstrated whereby, when forced to swim in a cylinder of water from which there is no escape, rats or mice will initially exhibit vigorous, escape-oriented, activity but eventually only make those minimal movements necessary to keep their heads above water. The test was shown to be sensitive to a range of drugs with known therapeutic activity against depression, some of which had not previously shown efficacy in the existing behavioural models.

Mice were individually placed in a cylinder (Height=24.5 cm, Diameter=19.5 cm) containing 13.5 cm water (22° C.) from which they could not escape. The mice were placed in the water for 6 min and the duration of immobility during the last 4 min was measured. Ten mice were studied per group. Rosemary extract (dissolved in corn oil) was evaluated at 272, 409 and 545 mg/kg body weight, administered orally 24, 5, and 1 h before the test, and compared with a vehicle-treated control group. Imipramine (32 mg/kg, p.o.; dissolved in distilled water), administered under the same experimental conditions, was used as reference substance. Data were analysed by comparing all groups using one-way Analysis of Variance (ANOVA) and the Bonferroni post-hoc test.

TABLE 2
Effects of rosemary extract (RE) and imipramine in
the FST in the mouse, following p.o. (−24, −5, −1 h)
administration.

| | DURATION OF IMMOBILITY | | | |
|---|---|---|---|---|
| | (as % total test time) | | | |
| TREATMENT (mg/kg) | | | | % change from |
| p.o., −24, −5, −1 h | mean ± S.D. | p value | | control |
| Vehicle | 43.76 ± 24.15 | — | | — |
| RE (272) | 28.96 ± 14.16 | 0.294 | NS | −34% |
| RE (409) | 15.13 ± 9.11 | 0.001 | ** | −65% |
| RE (545) | 19.86 ± 9.25 | 0.007 | ** | −55% |
| Imipramine (32) | 17.75 ± 9.92 | 0.004 | ** | −59% |

Data is shown as mean ± S.D. (n = 9-10) and as % change from control, where NS = non-significant, ** p < 0.01.

The results show that rosemary extract (409 and 545 mg/kg) demonstrated significant antidepressant-like activity, which was similar to that of imipramine.

### Example 5

### Effects of Rosemary Extract on Brain Levels of Serotonin and Noradrenaline (In Vivo Microdialysis in Rats)

Adult male Sprague Dawley rats were anaesthetised (chloral hydrate, 400 mg/kg i.p.), then a single microdialysis probe (BASi type MD2200, 2 mm membrane, 30,000 dalton cut-off) was implanted in the dorsal hippocampus, fixed in position with dental cement and perfused with artificial cerebrospinal fluid (aCSF) at 1 ml/min. Body temperature was maintained at 36° C., throughout all experiments. Extracellular serotonin (5-HT) and noradrenaline (NA) levels were determined by collection of perfusate samples every 15 min and assayed using high-performance liquid chromatography (HPLC) with electrochemical detection. The HPLC mobile phase (5-HT analysis: 0.5 mM EDTA, 0.1 M monochloroacetic acid, pH 3.1, 0.15 g/L sodium octyl sulphate, 5% acetonitrile, 0.7% tetrahydrofuran; NA analysis: 0.5 mM EDTA, 0.1 M NaH₂PO₄, pH 3.1, 1 mM sodium octyl sulphate, 6% acetonitrile) was pumped through the system at 70 μl/min. Monoamines were separated using a reverse-phase 1×100 mm ODS 3 μm microbore column with 5 μl injection loop and detected using an Epsilon electrochemical detector (BASi) with a glassy carbon electrode set at ⁺650 mV versus Ag/AgCl reference electrode. Dialysate peaks were identified by comparing peak elution times with reference standards and quantified according to measurement of peak area using linear regression analysis.

The first two perfusate samples collected after the start of microdialysis (−30 and −15 min) were used for baseline measurements, while sample collection was continued until 90 min after the last compound administration. Rosemary extract was suspended in corn oil and administered i.p. at 0 and 90 min (27 and 81 mg/kg, respectively).

The effect of each dose of the extract on the total amounts of serotonin and noradrenaline were calculated by estimation of the area-under-the-curve (AUC) over the sampling period following each dose administration. Effects of rosemary extract, calculated as percentage of basal values, were compared to a vehicle control group, using a two-way ANOVA followed by post-hoc comparisons at individual doses using the Bonferroni t-test (Graphpad Prism).

TABLE 3
Effect of rosemary extract (RE) on cumulative 5-HT and NA release,
measured as AUC during the 90 min following
administration of each dose.

| | |
|---|---|
| TREATMENT (mg/kg, i.p.) | AUC (% basal) |

| | | | |
|---|---|---|---|
| Time of injection | Substance | 5-HT | NA |

| | | | | | |
|---|---|---|---|---|---|
| 0 min | Vehicle | 80% | — | 96% | — |
| | RE (27) | 86% | NS | 101% | NS |
| 90 min | Vehicle | 64% | — | 96% | — |
| | RE (81) | 80% | NS | 106% | * |

Data is shown as % basal values;
NS = non-significant,
* p < 0.05, compared with control values.

Following each dose, the 5-HT level transiently increased, but then returned to values similar to the vehicle. However, there was no significant effect of treatment, as calculated over the entire treatment time (F(1,102)=2.689; p>0.05) and no significant effect of treatment, when calculated as AUC after each dose (F(1,15)=1.02; p>0.05). Following the lower dose there was no change in NA level, but there was a small (approximately 10%), yet sustained, increase in NA level following the higher dose. There was thus a significant effect of treatment, both when calculated over the entire treatment time (F(1,128)=17.63; p<0.001), and when calculated as AUC after each dose (F(1,20)=6.84; p<0.05).

Thus, using in vivo microdialysis, the rosemary extract according to Example 1 demonstrated a tendency to increase brain extracellular serotonin levels, while inducing significant increases in extracellular noradrenaline.

### Example 6

### Preparation of a Soft Gelatine Capsule

A soft gelatine capsule may be prepared comprising the following ingredients:

| Ingredient | Amount per Capsule |
|---|---|
| Rosemary extract | 500 mg |
| Lecithin | 50 mg |
| Soy bean oil | 250 mg |

Two capsules per day for 3 months may be administered to a human adult for the treatment of mild chronic dysthymia.

### Example 7

### Preparation of a Soft Gelatine Capsule

A soft gelatine capsule may be prepared comprising the following ingredients:

| Ingredient | Amount per Capsule |
|---|---|
| Rosemary extract | 200 mg |
| Evening primrose oil | 300 mg |
| Vitamin B₆ | 100 mg |

One capsule per day, preferably during the second half of the menstrual cycle may be taken for 14 days for the treatment of premenstrual syndrome and premenstrual dysphoric disorder.

### Example 8

### Preparation of a Tablet

A tablet may be prepared comprising the following ingredients:

| Ingredient | Amount per tablet |
|---|---|
| Rosemary extract | 100 mg |
| Passion flower standardised extract | 150 mg |
| Green Tea Extract, e.g. TEAVIGO ® | 150 mg |
| from DSM Nutritional Products, | |
| Kaiseraugst, Switzerland | |

For general well-being, energising and stress alleviation, one tablet may be taken twice daily for 3 months.

### Example 9

### Preparation of an Instant Flavoured Soft Drink

| Ingredient | Amount [g] |
|---|---|
| Rosemary extract | 0.9 |
| Sucrose, fine powder | 922.7 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, | 0.4 |
| Kaiseraugst, Switzerland | |
| Total amount | 1000 |

All ingredients are blended and sieved through a 500 μm sieve. The resulting powder is put in an appropriate container and mixed in a tubular blender for at least 20 minutes. For preparing the drink, sufficient water is added to 125 g of the obtained mixed powder to make up to one litre of beverage.

The ready-to-drink soft drink contains ca. 30 mg rosemary extract per serving (250 ml). As a strengthener and for general well-being 2 servings per day (240 ml) may be drunk.

### Example 10

### Preparation of a Fortified Non-Baked Cereal Bar

| Ingredient | Amount [g] |
|---|---|
| Rosemary extract | 0.95 |
| Sugar | 114.55 |
| Water | 54.0 |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.5 |
| Hardened palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skimmed milk powder | 45.0 |
| Apple flavour 74863-33 | 2.0 |
| Citric acid | 5.0 |
| Total amount | 1000 |

Rosemary extract is premixed with skimmed milk powder and placed in a planetary bowl mixer. Cornflakes and rice crispies are added and the total is mixed gently. Then the dried and cut apples are added. In a first cooking pot sugar, water and salt are mixed in the amounts given above (solution 1). In a second cooking pot glucose, invert- and sorbitol-syrup are mixed in the amounts given above (solution 2). A mixture of baking fat, palm kernel fat, lecithin and emulsifier is the fat phase. Solution 1 is heated to 110° C. Solution 2 is heated to 113° C. and then cooled in a cold water bath. Afterwards solutions 1 and 2 are combined. The fat phase is melted at 75° C. in a water bath, then added to the combined mixture of solutions 1 and 2. Apple flavour and citric acid are added to the liquid sugar-fat mix. The liquid mass is added to the dry ingredients and mixed well in the planetary bowl mixer. The mass is put on a marble plate and rolled to the desired thickness, then cooled down to room temperature and cut into pieces. The non-baked cereal bar contains ca. 25 mg rosemary extract per serving (30 g). For general well-being and energising 1-2 cereal bars may be eaten per day.

### Example 11

### Dry Dog Feed Containing Rosemary Extract for Relieving Stress and Revitalising the Dog

Commercial basal diet for dogs (e.g. Mera Dog “Brocken”, MERA-Tiernahrung GmbH, Marienstraβe 80-84, D-47625 Kevelaer-Wetten, Germany) is sprayed with a solution of rosemary extract in an amount sufficient to administer to a subject a daily dose of 50 mg per kg body weight based on the weight of the rosemary extract. The food composition is dried to contain dry matter of about 90% by weight. For an average dog of 10 kg body weight to consume approx. 200 g dry feed per day, the dog food contains approx. 2500 mg rosemary extract/kg food. For heavier dogs, the feed mix is prepared accordingly.

### Example 12

### Wet Cat Food Containing Rosemary Extract

Commercial basal diet for cats (e.g. Happy Cat “Adult”, Tierfeinnahrung, Südliche Hauptstraβe 38, D-86517 Wehringen, Germany) is mixed with a solution of rosemary extract in an amount sufficient to administer to a subject a daily dose of 100 mg per kg body weight based on the weight of the dried rosemary extract. For an average cat of 5 kg of body weight to consume approx. 400 g of wet food, the cat food contains 1250 mg/kg of rosemary extract. The food composition is dried to contain dry matter of about 90% by weight.

### Example 13

### Dog Treats Containing Rosemary Extract

Commercial dog treats (e.g. Mera Dog “Biscuit” for dogs as supplied by Mera Tiernahrung GmbH, Marienstrasse 80-84, 47625 Kevelaer-Wetten, Germany) are sprayed with a solution of rosemary extract in an amount sufficient to administer to the treats 5-50 mg per g treats based on the weight of the dried rosemary extract. The food composition is dried to contain dry matter of about 90% by weight.

### Example 14

### Cat Treats Containing Rosemary Extract

Commercial cat treats (e.g. Whiskas Dentabits for cats as supplied by Whiskas, Masterfoods GmbH, Eitzer Str. 215, 27283 Verden/Aller, Germany) are sprayed with a solution of rosemary extract in an amount sufficient to administer to the treats 5-50 mg per g treats based on the weight of the dried rosemary extract. The food composition is dried to contain dry matter of about 90% by weight.

1. An acetone extract of rosemary leaves for use as medicament for the treatment of a disorder connected to reduced neurotransmission.

2. The acetone extract according to claim 1 manufactured by a process comprising the following steps:
a) grinding dried leaves of Rosmarinus officinalis;
b) extracting the leaves of step a) with acetone to obtain an acetone extract;
c) optionally filtrating the acetone extract;
d) concentrating the acetone extract by (partly, preferably nearly completely) removing the acetone;
e) optionally spray-drying the thus concentrated acetone extract.

3. The acetone extract according to claim 1 wherein the acetone extract contains rosmanol (preferably in the range of from 0.05 to 3 weight-%), carnosol (preferably in the range of from 0.5 to 7 weight-%), carnosic acid (preferably in the range of from 20 to 65 weight-%) and carnosic acid 12-methyl ether (preferably in the range of from 1 to 20 weight-%; all amounts based on the total weight of the acetone extract) and is preferably manufactured by a process according to claim 2.

4. The rosemary extract according to claim 1 for use as antidepressant, mood/vitality improver, stress reliever, condition improver, reducer of anxiety, reducer of obsessive-compulsive behaviour, relaxant, sleep improver and/or insomnia alleviator.

5. Use of a rosemary extract according to claim 1 for the manufacture of a composition for the treatment of a disorder connected to reduced neurotransmission.

6. The use according to claim 5, wherein the composition is an antidepressant, mood/vitality improver, stress reliever, condition improver, reducer of anxiety, reducer of obsessive-compulsive behaviour, relaxant, sleep improver and/or insomnia alleviator.

7. A dietary composition containing a rosemary extract as defined in claim 1.

8. A pharmaceutical composition containing a rosemary extract as defined in claim 1 and a conventional pharmaceutical carrier.

9. A method for the treatment of a disorder connected to reduced neurotransmission in animals including humans, said method comprising administering an effective dose of a rosemary extract as defined in claim 1 to animals including humans which are in need thereof.

10. The method according to claim 9, wherein the animal is a human, a pet animal or a farm animal.
